(19) United States
(12) Patent Application Publication
(10) Pub. No.: US 2013/0066218 A1
(43) Pub. Date: Mar. 14, 2013

(54) APPARATUS FOR MEASURING PULSE AND METHOD OF OBTAINING PULSE INFORMATION USING THE SAME

(71) Applicant: Young Sung KIM, Seoul (KR)

(72) Inventor: Young Sung KIM, Seoul (KR)

(73) Assignee: Electronics and Telecommunications Research Institute, Daejeon (KR)

(21) Appl. No.: 13/605,371

(22) Filed: Sep. 6, 2012

(30) Foreign Application Priority Data
Sep. 8, 2011 (KR) 10-2011-0091024

(51) Int. Cl. A61B 5/0245

(52) U.S. Cl. 600/500

(57) ABSTRACT

A pulse measurement apparatus and a method of obtaining pulse information using the same are provided. The pulse measurement apparatus includes a frame, a first arm and a second arm installed in the frame so that the first and the second arms are rotated, and a sensor unit formed at one end of each of the first arm and the second arm, brought in contact with an inner wall within an ear of a user, and configured to detect pulse waves transferred through a blood vessel. The pulse measurement apparatus mounted on an ear and reduced in size can be provided, and a continuous and stable pulse wave signal can be measured using the pulse measurement apparatus.

### CROSS-REFERENCE TO RELATED APPLICATIONS

This application claims the benefit of priority of Korean Patent Application No. 10-2011-0091024 filed on Sep. 8, 2011, all of which is incorporated by reference in its entirety herein.

### BACKGROUND OF THE INVENTION

1. Field of the Invention

The present invention relates to an apparatus for measuring a pulse and a method of obtaining pulse information using the same and, more particularly, to an apparatus and method for detecting pulse waves transferred through a blood vessel within an ear by using a piezoelectric sensor and extracting a pulse based on the detected pulse waves.

2. Related Art

Most of pulse measurement apparatuses are being used in the following three fields. In the first field, the pulse measurement apparatus is used to monitor the state of a patient in a hospital or an ambulance. In this pulse measurement apparatus, an apparatus, such as an electrocardiogram or a sphygmograph for measuring a pulse using a non-invasive method, or an invasive pulse measurement apparatus for measuring a pulse through a pipe inserted into the blood vessel of a patient is chiefly used. Most of the apparatuses are easy to manipulate and may be used without being limited to a size.

In the second field, the pulse measurement apparatuses are used to recommend quantity of motion suitable the physical strength or age of a person who takes exercise. The pulse measurement apparatuses are constructed in connection with sports equipment. That is, sensors and a system for measuring a pulse are included inside or outside the sports equipment so that a pulse can be measured in real time during exercise. The pulse measurement apparatuses may be used in connection with sports equipment without being limited to a size.

In the third field, portable pulse measurement apparatuses are used to check a health state in daily life. The pulse measurement apparatuses are reduced in size and weight so that they may be worn on the body. For example, a pulse measurement sensor is embedded in a device or an accessory easy to carry on, such as a head set, an ear set, or a wristwatch, so that a pulse can be measured in daily life.

Meanwhile, people who measure a pulse using a portable pulse measurement apparatus in order to check their health state at normal times are recently increasing. A method of implementing the portable pulse measurement apparatus includes the following two representative methods.

The first method is to measure a pulse using a photo sensor. That is, when the human body transmits light, a peripheral vascular amount can be measured using the photo sensor and pulse waves can be extracted from the peripheral vascular amount. If the photo sensor is fixed to the human body, a pulse can be stably measured. However, a stable pulse wave signal is not provided in a situation having a great movement, such as exercise. Furthermore, the photo sensor is bulky in being attached to the human body and carried on in daily life, and it has great power consumption.

The second method is to measure a pulse using an air pressure sensor. That is, the air pressure sensor measures a pulse by detecting a change in the pressure of air from pulsation which is transferred to the inside of an ear or the periphery of the ear. The air pressure sensor is not used as an independent pulse measurement apparatus and must be embedded in a device, such as an ear set or a head set.

### SUMMARY OF THE INVENTION

An object of the present invention is to provide an apparatus mounted on an ear and configured to measure a pulse by using a pulse wave signal transferred to a blood vessel within an ear and a method using the same.

Another object of the present invention is to provide an apparatus and method for obtaining a stable pulse wave signal by finding a point within an ear at which the pulse wave signal is easy to detect and fixing the point.

In an embodiment of the present invention, a pulse measurement apparatus is provided. The pulse measurement apparatus includes a frame, a first arm and a second arm installed in the frame so that the first and the second arms are rotated, and a sensor unit formed at one end of each of the first arm and the second arm, brought in contact with an inner wall within an ear of a user, and configured to detect pulse waves transferred through a blood vessel.

The sensor unit may be formed of a piezoelectric sensor.

The piezoelectric sensor may have a film form.

The piezoelectric sensor may detect the pulse waves and convert the detected pulse waves into an electrical signal.

The first arm and the second arm may be coupled at a coupling unit in such a way as to cross each other and are rotated and moved around the coupling unit.

The pulse measurement apparatus may further include an elastic member provided at the other ends of the first arm and the second arm and configured to provide elastic force in a direction that widens an interval between the one ends of the first arm and the second arm.

The sensor unit may be closely adhered to the inner wall within the ear of the user by the elastic member in the state in which the sensor unit has been inserted into the inside of the ear of the user.

The coupling unit may be configured to have a spherical shape and, may be inserted into and installed in the hollow of the frame.

The first arm and the second arm may be made of silicon.

In the present embodiment, the pulse measurement apparatus may further include a processing unit for extracting pulse information of the user based on the pulse waves detected by the sensor unit and, a wireless transmission unit for wirelessly transmitting the pulse information of the processing unit to an external reception apparatus.

The processing unit may include a signal processing unit for amplifying an electrical signal detected by the sensor unit, removing noise from the amplified electrical signal, and generating a pulse wave signal and, a central processing unit for extracting a pulse by detecting a period from the pulse wave signal generated from the signal processing unit.

In another embodiment of the present invention, a method of obtaining pulse information is provided. The method includes inserting a head into the inside of an ear of a user and closely adhering the head to an inner wall within the ear of the user, detecting a pulse wave signal transferred through a blood vessel passing through the inside of the ear, from the head closely adhered to the inner wall within the ear, extracting pulse information from the detected pulse wave signal, and wirelessly transmitting the extracted pulse information to an external reception apparatus.

The head may include a piezoelectric sensor, and the piezoelectric sensor may be installed in the outside of the head.

Detecting the pulse wave signal may include detecting pulse waves and converting the detected pulse waves into an electrical signal by using the piezoelectric sensor.

Extracting the pulse information may include amplifying the electrical signal, removing noise from the amplified electrical signal, and generating the pulse wave signal and extracting the pulse information by detecting a period from the generated pulse wave signal.

### BRIEF DESCRIPTION OF THE DRAWINGS

FIG. 1 is a perspective view showing a pulse measurement apparatus 10 according to an embodiment of the present invention.

FIG. 2 is a block diagram showing an internal construction of the pulse measurement apparatus 10 according to an embodiment of the present invention.

FIGS. 3A and 3B are diagrams schematically illustrating an embodiment in which a head 100 is closely adhered to and separated from the inner wall within the ear according to the present invention.

FIG. 4 is a flowchart illustrating a method of obtaining pulse information by measuring a pulse using the pulse measurement apparatus 10 according to an embodiment of the present invention.

### DESCRIPTION OF EXEMPLARY EMBODIMENTS

Hereinafter, embodiments of the present invention will be described in detail with reference to the accompanying drawings so that they can be readily implemented by those skilled in the art.

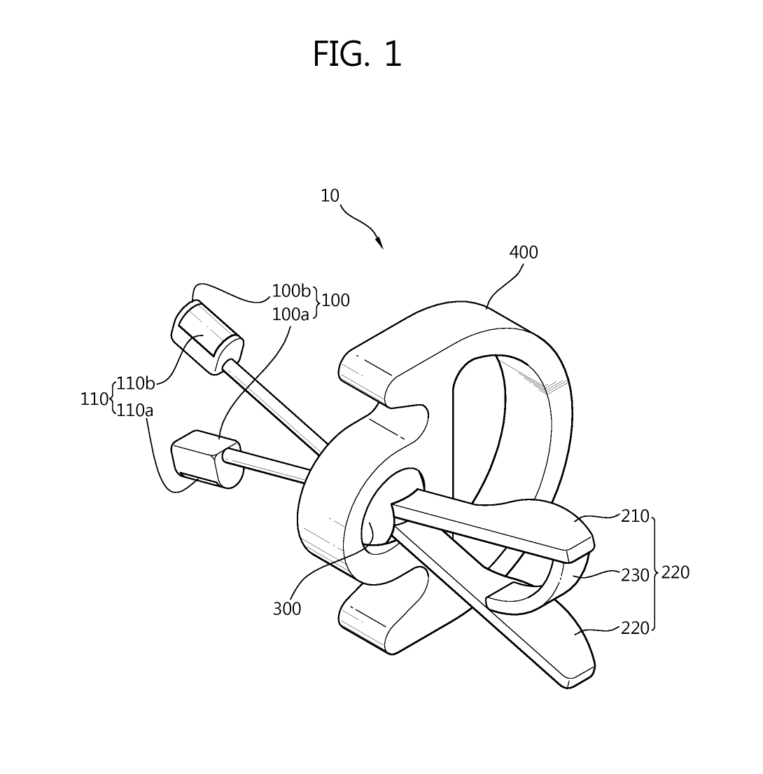

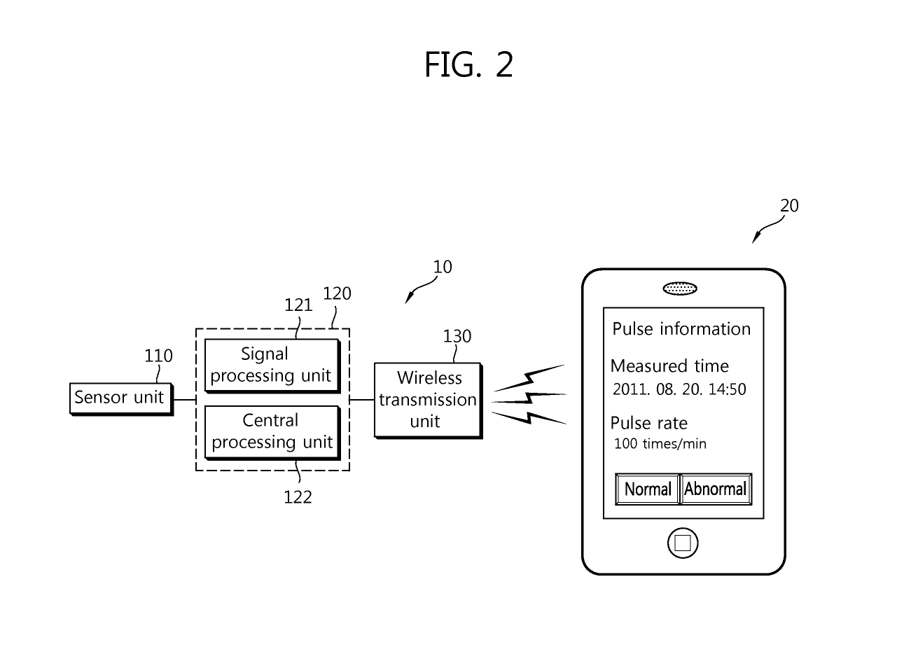

FIG. 1 is a perspective view showing a pulse measurement apparatus 10 according to an embodiment of the present invention, and FIG. 2 is a block diagram showing an internal construction of the pulse measurement apparatus 10 according to an embodiment of the present invention.

As shown in FIG. 1, the pulse measurement apparatus 10 according to the present invention includes a head 100, an arm 200, and a frame 300. The pulse measurement apparatus 10 is mounted on an ear of a user and configured to measure a pulse.

The frame 300 is a fixing shaft for fixing the pulse measurement apparatus 10 to an ear of a user and is constructed in a form which may be fixed to the earhole. Furthermore, the frame 300 may be made of flexible and elastic material and may be configured to be suitable for a different shape of an ear for each person. The frame 300 may be also configured to be fixed to an auricle or an ear in addition to the earhole.

Furthermore, the arm 200 is installed in the frame 300. The arm 200 may be rotated and moved. For example, the frame 300 may be configured to have a hollow shape, and the arm 200 may be inserted into the hollow.

The arm 200 includes a first arm 210 and a second arm 220, each having a pole shape. The first arm 210 and the second arm 220 cross each other at a coupling unit 240, and they are coupled at the coupling unit 240. The coupling unit 240 may be formed to have a spherical shape and inserted into the hollow part of the frame 300. Accordingly, the first arm 210 and the second arm 220 may be installed in the frame 300 and may be rotated and moved around the coupling unit 240 with the degree of freedom of 30° or more up and down and left and down.

Meanwhile, the head 100 is installed at one end of each of the first arm 210 and the second arm 220. Here, the head 100 may be formed of a pair of heads. That is, one head 100a may be installed at one end of the first arm 210, and the other head 100b may be installed at one end of the second arm 220.

Furthermore, an elastic member 230 may be installed at the other end of the first arm 210 or the second arm 220. The elastic member 230 may be formed of an elastic member, and it provides elastic force for separating an interval between the ends of the first arm 210 and the second arm 220 from each other.

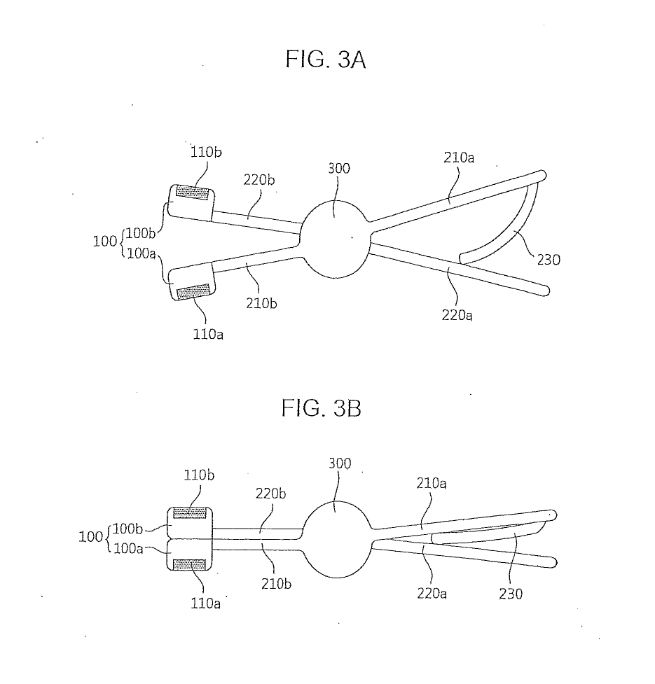

More particularly, as shown in FIG. 3A, an elastic member 230 installed in a first arm 210a on one side applies force to a second arm 220a on one side in a direction that separates the second arm 220a from the first arm 210a. Accordingly, an interval between the ends of the first arm 210b and the second arm 220b on the other side is widened, and heads 100a and 100b installed in the first arm 210b and the second arm 220b, respectively, may maintain the widened state.

At this time, when force applies to and presses the sides of the first arm 210a and the second arm 220a, the elastic member 230 is deformed. For example, as shown in FIG. 3B, the elastic member 230 is compressed and thus an interval between the ends of the first arm 210b and the second arm 220b is narrowed. Accordingly, the heads 100a and 100b installed at the ends of the first arm 210b and the second arm 220b becomes from the widened state to a narrowed state.

Furthermore, when force applied to the sides of the first arm 210a and the second arm 220a is removed, the elastic member 230 has elastic force that the elastic member 230 tries to return to its original state. As shown in FIG. 3A, an interval between the ends of the first arm 210b and the second arm 220b is widened by the elastic force of the elastic member 230, and thus the heads 100a and 100b installed in the first arm 210b and the second arm 220b have the widened state again.

That is, when an interval between the pair of heads 100a and 100b is widened by the elastic member 230, the heads 100a and 100b may be closely adhered to an inner wall within an ear. When the elastic member 230 is deformed by force, the pair of heads 100a and 100b may be separated from the inner wall within the ear. Furthermore, the degree that the heads 100a and 100b are closely adhered to the inner wall within the ear may be controlled by controlling force applied to the elastic member 230 by taking the sensitivity of the skin within an ear of a user into consideration.

Referring to FIG. 1, the head 100 is a part that is inserted into the inside of an ear of a user and brought in contact with an inner wall within the ear. The head 100 is implemented using a structure and material into which a shape of the inside of the ear is taken into consideration so that the head 100 is closely adhered and fixed to the inner wall within the ear. For example, the head 100 may be made of flexible material, such as silicon, so that the head 100 is controlled suitably for a shape of the inside of an ear of a person.

Furthermore, the head 100 is equipped with a sensor unit 110 inserted into the inside of an ear and configured to detect pulse waves transferred from an inner wall within the ear. The pulse wave is a wave motion that is propagated from the heat along a blood vessel. The wave motion is transferred to a blood vessel existing within an ear. Accordingly, the sensor unit 110 may have a structure that directly comes in contact with an inner wall within the ear in order to easily detect the pulse wave transferred to the blood vessel within the ear. For example, the sensor unit 110 may be installed in the outside of the head 100.

Meanwhile, in order for the pulse measurement apparatus 10 according to the present invention to detect a pulse wave from an inner wall within an ear, first, the head 100 equipped with the sensor unit 110 is inserted into the inside of the ear and then placed at a point adjacent to a vein or the artery within the ear. That is, the head 100 installed at one end of the arm 200 is inserted up to a point close to the middle ear via the external ear. Here, a total of two curves exist from the external ear, that is, the start point of the earhole, to the middle ear where the eardrum is placed. The first curve has a different length and shape for each person. For this reason, the head 100 and the arm 200 may be made of flexible material, for example, silicon so that the head 100 is inserted up to the point close to the middle ear irrespective of the curve shape.

When the head 100 is inserted up to the point close to the middle ear, the sensor unit 110 is closely adhered to the inner wall within the ear which is closed to a vein or the artery by using the arm 200. As described above, the arm 200 is installed in the frame 300 and can be moved around the coupling unit 240 with the degree of freedom of 30° or more. Accordingly, the position of the head 100 installed at one end of the arm 200 may be adjusted so that it is placed at a desired place of the inner wall within the ear. Furthermore, the head 100 may be closely adhered to the inner wall within the ear by using the elastic member 230 installed in the arm 200.

Next, in order for the head 100 to keep closely adhered to the inner wall within the ear, the frame 300 is fixed to the earhole. As described above with reference to FIGS. 3A and 3B, the position of the head 100 may be adjusted again by controlling force applied to the elastic member 230 and rotating and moving the arm 200. The head 100 can keep closely adhered and fixed to the inner wall within the ear by the frame 300. Accordingly, even in case of great motion, such as exercise, the sensor unit 110 may obtain a stable pulse wave signal.

An internal construction of the pulse measurement apparatus 10 according to an embodiment of the present invention is described below with reference to FIG. 2.

The pulse measurement apparatus 10 according to the present invention includes the sensor unit 110 for detecting pulse waves, a processing unit 120 for extracting pulse information from the detected pulse wave, and a wireless transmission unit 130 for wirelessly transmitting the extracted pulse information.

The sensor unit 110 detects pulse waves transferred through a blood vessel within an ear of a user. To this end, a piezoelectric sensor may be used as the sensor unit 110. The piezoelectric sensor is based on a piezoelectric effect that generates voltage when vibration or force is applied thereto. The piezoelectric sensor detects pulse waves transferred through a blood vessel within an ear and converts the detected pulse waves into an electrical signal. For example, a film type piezoelectric sensor of less than 0.1 mm in thickness may be used as the sensor unit 110. The film type piezoelectric sensor may be adhered to the outside of the head 100 so that the sensor unit 110 is closely adhered to the inner wall within the ear.

The processing unit 120 receives the electrical signal from the sensor unit 110 and extracts pulse information from the electrical signal. The processing unit 120 may include a signal processing unit 121 and a central processing unit 122.

The signal processing unit 121 receives the electrical signal converted by the piezoelectric sensor, amplifies the received electrical signal, removes noise from the amplified signal, and generates a necessary pulse wave signal. The central processing unit 122 converts the pulse wave signal, generated from the signal processing unit 121, into a digital signal and extracts a pulse by detecting the period of the pulse wave signal form the digital signal.

The wireless transmission unit 130 transmits the pulse information, extracted by the processing unit 120, to an external reception apparatus 20 through wireless communication. For example, the wireless transmission unit 130 may transmit the pulse information to the external reception apparatus 20 (e.g., a smart phone, a PDA, or a notebook computer) through wireless communication, such as Bluetooth, Zig-Bee, or WiBro. Furthermore, the reception apparatus 20 may receive the pulse information and display the received pulse information on a screen. That is, a user may measure a pulse by using the pulse measurement apparatus 10 according to the present invention and then check pulse information by using the reception apparatus 20.

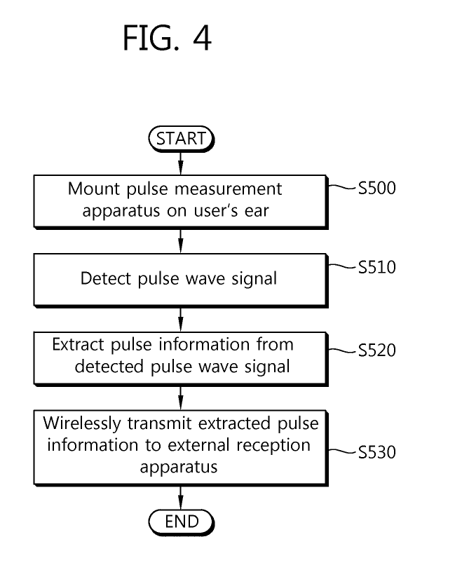

FIG. 4 is a flowchart illustrating a method of obtaining pulse information by measuring a pulse using the pulse measurement apparatus 10 according to an embodiment of the present invention.

Referring to FIG. 4, first, a user mounts the pulse measurement apparatus 10 on his ear in order to measure his pulse at step S500. For example, the user may insert the head 100 equipped with the sensor unit 110, up to a point close to the artery or a vein within the ear, closely adhere the head to an inner wall within the ear, and fix the pulse measurement apparatus 10 to the inner wall by using the frame 300.

Next, a pulse wave signal is detected by using the pulse measurement apparatus 10 mounted on the ear at step S510. For example, the sensor unit 110 formed of a film type piezoelectric sensor having a thin thickness may be adhered to the outside of the head 100 and closely adhered to the inner wall within the ear. Here, the piezoelectric sensor may detect the pulse wave signal transferred from the inner wall within the ear and convert the detected pulse wave signal into an electrical signal.

The pulse measurement apparatus 10 extracts pulse information from the detected pulse wave signal at step S520. That is, the pulse measurement apparatus 10 extracts the pulse information using the electrical signal converted by the sensor unit 110. For example, as described above in connection with the processing unit 120 of FIG. 2, the processing unit 120 amplifies the converted electrical signal having weak electricity, removes noise from the amplified signal, and generates a necessary pulse wave signal. Next, the generated pulse wave signal is converted into a digital signal and a pulse is extracted by detecting a period from the converted digital signal.

Next, the pulse measurement apparatus 10 wirelessly transmits the extracted pulse information to the external reception apparatus 20 at step S530. For example, the pulse measurement apparatus 10 including the wireless transmission unit 130 may transmit the pulse information the external reception apparatus 20 through wireless communication. The reception apparatus 20 may receive the pulse information and output the pulse information on a screen. Accordingly, the pulse measurement apparatus 10 according to the present invention is a small-sized and independent system mounted on an ear and configured to measure a pulse. When the pulse measurement apparatus 10 provides pulse information at the request of a user in daily life, the user may check the pulse information.

In accordance with the present invention, the pulse measurement apparatus mounted on an ear and reduced in size can be provided, and pulse waves transferred through a blood vessel within an ear can be detected using the pulse measurement apparatus. Furthermore, a pulse may be measured using the detected pulse wave.

In accordance with the present invention, a point within an ear at which a pulse wave signal is easy to detect may be designated and fixed. Accordingly, a stable pulse wave signal can be obtained.

The embodiments of the pulse measurement apparatus and the method of obtaining pulse information using the same according to the present invention have been described above. However, a person having ordinary skill in the art may modify and change the embodiments of the present invention in various ways by adding, changing, deleting, or supplementing elements within the scope of the present invention. Furthermore, it is evident that those modifications and changes fall within the scope of the present invention.

What is claimed is:

1. A pulse measurement apparatus, comprising:
a frame;
a first arm and a second arm installed in the frame so that the first and the second arms are rotated; and
a sensor unit formed at one end of each of the first arm and the second arm, brought in contact with an inner wall within an ear of a user, and configured to detect pulse waves transferred through a blood vessel.

2. The pulse measurement apparatus of claim 1, wherein the sensor unit is formed of a piezoelectric sensor.

3. The pulse measurement apparatus of claim 2, wherein the piezoelectric sensor has a film form.

4. The pulse measurement apparatus of claim 2, wherein the piezoelectric sensor detects the pulse waves and converts the detected pulse waves into an electrical signal.

5. The pulse measurement apparatus of claim 1, wherein the first arm and the second arm are coupled at a coupling unit in such a way as to cross each other and are rotated and moved around the coupling unit.

6. The pulse measurement apparatus of claim 5, further comprising an elastic member provided at other ends of the first arm and the second arm and configured to provide elastic force in a direction that widens an interval between the one ends of the first arm and the second arm.

7. The pulse measurement apparatus of claim 6, wherein the sensor unit is closely adhered to the inner wall within the ear of the user by the elastic member in the state in which the sensor unit has been inserted into the inside of the ear of the user.

8. The pulse measurement apparatus of claim 5, wherein the coupling unit is configured to have a spherical shape and, is inserted into and installed in a hollow of the frame.

9. The pulse measurement apparatus of claim 1, wherein the first arm and the second arm are made of silicon.

10. The pulse measurement apparatus of claim 1, further comprising a processing unit for extracting pulse information of the user based on the pulse waves detected by the sensor unit.

11. The pulse measurement apparatus of claim 10, further comprising a wireless transmission unit for wirelessly transmitting the pulse information extracted by the processing unit to an external reception apparatus.

12. The pulse measurement apparatus of claim 10, wherein the processing unit comprises:
a signal processing unit for amplifying an electrical signal detected by the sensor unit, removing noise from the amplified electrical signal, and generating a pulse wave signal; and
a central processing unit for extracting a pulse by detecting a period from the pulse wave signal generated from the signal processing unit.

13. A method of obtaining pulse information, the method comprising:
inserting a head into an inside of an ear of a user and closely adhering the head to an inner wall within the ear of the user;
detecting a pulse wave signal transferred through a blood vessel passing through the inside of the ear, from the head closely adhered to the inner wall within the ear;
extracting pulse information from the detected pulse wave signal; and
wirelessly transmitting the extracted pulse information to an external reception apparatus.

14. The method of claim 13, wherein the head comprises a piezoelectric sensor, and the piezoelectric sensor is installed in an outside of the head.

15. The method of claim 14, wherein detecting the pulse wave signal comprises detecting pulse waves and converting the detected pulse waves into an electrical signal by using the piezoelectric sensor.

16. The of claim 15, wherein extracting the pulse information comprises:
amplifying the electrical signal, removing noise from the amplified electrical signal, and generating the pulse wave signal; and
extracting the pulse information by detecting a period from the generated pulse wave signal.
